(19) United States
(12) Patent Application Publication
(10) Pub. No.: US 2005/0064516 A1
(43) Pub. Date: Mar. 24, 2005

(54) Biological markers for diagnosing multiple sclerosis

(76) Inventors: Aaron B. Kantor, San Carlos, CA (US); Christopher H. Becker, Palo Alto, CA (US)

Correspondence Address: SWANSON & BRATSCHUN L.L.C., 1745 SHEA CENTER DRIVE, SUITE 330, HIGHLANDS RANCH, CO, 80129, US

(21) Appl. No.: 10/946,359

(22) Filed: Sep. 20, 2004

Related U.S. Application Data
(60) Provisional application No. 60/504,468, filed on Sep. 18, 2003.

(51) Int. Cl. G01N 33/53

(52) U.S. Cl. 435/7.1

(57) ABSTRACT

Biological markers for multiple sclerosis, and their use in the diagnosis and clinical applications of the disease, are described.

### CROSS-REFERENCE TO RELATED APPLICATIONS

This application claims the benefit, under 35 U.S.C. § 119, of U.S. Provisional Patent Application Ser. No. 60/504,468, entitled “Biological Markers For Diagnosing Multiple Sclerosis,” filed September 18, 2003, and incorporated by reference herein in its entirety.

### FIELD OF THE INVENTION

The present invention relates to biological markers for Multiple Sclerosis. More specifically, the present invention relates to the use of such markers to diagnose Multiple Sclerosis, monitor progression of the disease, evaluate therapeutic interventions, and screen candidate drugs in a clinical or preclinical trial.

### BACKGROUND OF INVENTION

Multiple Sclerosis (MS) is the most common autoimmune disease involving the nervous system. The disease affects twice as many women as it does men. There are 350,000 persons affected with MS in the US with more than 10,000 new cases reported each year. Worldwide, MS affects nearly 2.5 million individuals. There is a high economic burden associated with the disease. The total annual cost for all people with MS in the US has been estimated to be more than $9 billion dollars. Whetten-Goldstein, K., Sloan, F. A., Goldstein, L. B. & Kulas, E. D. A comprehensive assessment of the cost of multiple sclerosis in the United States. Mult Scler 4, 419-425 (1998).

Clinically, the disease can be broadly divided into a relapsing remitting form characterized by a series of exacerbations that result in varying degrees of disability from which the patient recovers, and a progressive form in which the patient does not experience exacerbations, but instead reports a gradual decline. A relapsing-remitting onset is observed in 85-90% of patients. The course of the disease in about 40% of relapsing-remitting patients ultimately changes to a progressive form.

A well-demarked area of myelin loss, known as a “demyelinated plaque,” is the hallmark of the disease. Symptoms are believed to occur from axonal demyelination that inhibits or blocks conduction. Plaques may be found throughout the brain and spinal cord. Inflammatory cells are seen at the edges of the plaque and scattered throughout the white matter. Amelioration of symptoms has been attributed to partial remyelination and resolution of inflammation. Based on accumulating data from immunological studies of MS patients and a wealth of animal model data, autoimmune dysregulation has been viewed as the major contributor to tissue damage.

The current model of MS immunopathology suggests that autoreactive T cells within the periphery become activated. Noseworthy, J. H., Lucchinetti, C., Rodriguez, M. & Weinshenker, B. G. Multiple sclerosis. N Engl J Med 343, 938-952 (2000). Viral infection, bacterial lipopolysaccharides, superantigens, reactive metabolites, and metabolic stress may facilitate activation. Activated T cells express up-regulated levels of adhesion molecules and are able to migrate across the blood-brain barrier much more efficiently than naive, resting T cells. Extravasation across the blood-brain barrier is thought to involve a sequence of overlapping molecular interactions between inducible ligand-receptor pairs on the surface of the migrating cell and the endothelial barrier. Selective expression of adhesion molecules, chemokines and chemokine receptors and matrix metalloproteinases are likely to be important in mediating the transmigration of effector cells across the blood-brain barrier and into the central nervous system (CNS) perivascular tissue in demyelinating diseases.

Chemokines can enhance immune cell migration through direct chemoattraction and by activating leukocyte integrins to bind their adhesion receptors on endothelial cells. An increase in pro-inflammatory chemokines is associated with demyelination in MS. Further release of local cytokines, chemokines and matrix metalloproteinases may support the recruitment of subsequent waves of infiltrating effector cells, including T cells, monocytes and B cells. Invading autoreactive T cells can then become reactivated upon encounter with their cognate antigen in the CNS, thereby supporting local inflammation. Mechanisms of myelin destruction and axonal damage are likely to be multiple and include direct effects of pro-inflammatory cytokines, oxygen radicals and complement fixing antibodies, antigen specific and non-specific cytotoxicity, and apoptosis. Activation of resident CNS glial cells, such as microglia, may provide the basis for the generation or maintenance of pathologic responses, even in the absence of further infiltration of exogenous inflammatory cells.

An abnormal humoral immune response has also been well described in MS patients. See, e.g., Cross, A. H., Trotter, J. L. & Lyons, J. B cells and antibodies in CNS demyelinating disease. J Neuroimmunol 112, 1-14 (2001). A renewed interest in the possible contribution of B cells to MS immunopathology has been sparked with more recent MS pathological studies that indicate autoantibodies against a specific myelin protein may mediate target membrane damage in central nervous system demyelinating disease. Genain, C. P., Cannella, B., Hauser, S. L. & Raine, C. S. Identification of autoantibodies associated with myelin damage in multiple sclerosis. Nat Med 5, 170-175 (1999). Studies of the humoral immune response in MS have focused on the intrathecal immunoglobulin and the antibody response to brain antigens in the periphery. Immunolgobulin oligoclonal bands can be detected in more than 90% of patients with clinically definite MS. Thompson, E. J., Kaufmann, P., Shortman, R. C., Rudge, P. & McDonald, W. I. Oligoclonal immunoglobulins and plasma cells in spinal fluid of patients with multiple sclerosis. Br Med J 1, 16-17 (1979).

Substantial effort has been invested in the elucidation of the antigenic specificity of MS CSF immunoglobulin. Much of the earlier research in this field focused on exogenous antigens. However, these studies revealed the viral and bacterial antibodies constitute only a minor fraction of the elevated MS CSF immunoglobulin. Vartdal, F. & Vandvik, B. Multiple sclerosis. Electrofocused “bands” of oligoclonal CSF IgG do not carry antibody activity against measles, varicella-zoster or rotaviruses. J Neurol Sci 54, 99-107 (1982). Conversely, in many other inflammatory neurological diseases, antibodies directed against causative exogenous antigen account for much of the intrathecal immunoglobulin. In MS, however, the driving mechanism behind the Ig elevation has not been successfully assigned to an exogenous antigen. The possibility that CSF immunoglobulin in MS patients is generated as a response to myelin self-antigens has also been considered. For example, antibodies specific for myelin basic protein (MBP), proteolipid protein (PLP), myelin associated glycoprotein (MAG), transaldoase (TAL) and myelin oligodendrocyte glycoprotein (MOG) have been identified in the CSF of patients with MS.

The pathogenic mechanisms of MS may not be limited to autoimmunity. Hemmer, B., Archelos, J. J. & Hartung, H. P. New concepts in the immunopathogenesis of multiple sclerosis. Nat Rev Neurosci 3, 291-301 (2002). Demyelination may occur through many proposed mechanisms: Fas/Fas ligand interactions, toxic cytokines, reactive oxygen species, antibody dependent cellular toxicity and metabolic instability of oligodendrocytes. In addition, axonal damage is increasingly recognized as a prominent pathological feature in MS lesions as well as in normal appearing white matter in MS brains. Whereas these observations do not preclude the role of inflammatory demyelination in MS pathogenesis, axonal compromise may predate the inflammatory lesions, raising the possibility that an independent axonal pathology may contribute to the primary pathobiology of the disease. Studies of the mechanisms of axonal damage and neurodegeneration in MS are in their infancy. However, axonal damage may determine clinical outcome to a large extent. CNS tissue destruction markers would be useful not only for inflammatory demyelination but for neurodegenerative processes in MS. Isoprostane and N-acetylaspartate are two examples of such putative markers. Greco, A., Minghetti, L. & Levi, G. Isoprostanes, novel markers of oxidative injury, help understanding the pathogenesis of neurodegenerative diseases. Neurochem Res 25, 1357-1364 (2000); Gonen, O. et al. Total brain N-acetylaspartate: a new measure of disease load in MS. Neurology 54, 15-19 (2000).

An additional layer of complexity is added when considering the diversity of the disease within and among individuals. There is significant heterogeneity in clinical course, neuroradiological appearance of the lesions, involvement of susceptibility gene loci, and response to therapy. The spectrum of clinical MS spans several distinct pathophysiologic processes. Alternatively or (more likely) in combination, the clinical and pathological heterogeneity may reflect the diversity of unique host attributes. Neuropathological characterization of MS lesions reveals that they can be classified into at least four subtypes. Lucchinetti, C. et al. Heterogeneity of multiple sclerosis lesions: implications for the pathogenesis of demyelination. Ann Neurol 47, 707-717 (2000). However, the lesional profiles identified are conserved within individual patients. This indicates that different pathogenic pathways may be followed in different patients. Markers that differentiate such subtypes will be important in the design of therapeutic strategies.

MS is a systemic disease in terms of its autoimmune pathogenesis and a compartmental disease in as much as the end-organ damage is in the CNS. Thus, biomarkers of the disease would most likely be found in the CSF that bathes the brain, as well as in other more easily obtainable fluids, such as serum or urine, that are reflective of systemic disease. While CSF is not readily obtainable, especially for serial analysis, biomarkers found in CSF may guide the development of sensitive assays to enable detection of the candidate biomarkers in other fluids.

The availability of treatments that favorably impact the early course of MS underscores the importance of timely and accurate diagnosis. Currently, the diagnosis of MS is time consuming, expensive and uncertain especially in the early stages of disease. MRI has significantly improved diagnosis, but the correlation of MRI measures with clinical disability and outcome continues to be investigated. MRI has also been used to assess MS disease activity, disease burden and the dynamic evolution of these parameters over time. Bourdette, D., Antel, J., McFarland, H. & Montgomery, E., Jr. Monitoring relapsing remitting MS patients. J Neuroimmunol 98, 16-21 (1999). Serial MRI studies have unequivocally demonstrated that clinically apparent changes reflect only a minor component of disease activity. Overall MRI is limited in its ability to provide specific information about pathology in MS. In the absence of a specific defining assay, the diagnosis of MS continues to be predicated on the clinical history and neurological exam, though use of the MRI has had a major impact on early diagnosis. See McDonald, W. I.; Compston, A.; Edan, G.; et al. Recommended diagnostic criteria for multiple sclerosis: Guidelines from the International Panel on the Diagnosis of Multiple Sclerosis. Ann Neurol 50, 121-127 (2001).

Laboratory tests add important information. The CSF of patients with MS typically shows normal glucose, a few lymphocytes, normal to mildly elevated total protein, and immunoglobulin oligoclonal bands. Although often absent early in the disease, oligoclonal bands can eventually be detected in over 90% of patients with MS. McLean, B. N., Luxton, R. W. & Thompson, E. J. A study of immunoglobulin G in the cerebrospinal fluid of 1007 patients with suspected neurological disease using isoelectric focusing and the Log IgG-Index. A comparison and diagnostic applications. Brain 113, 1269-1289 (1990). Oligoclonal bands are also present in several other infectious, inflammatory and lymphoproliferative disorders. The use of MRI to monitor MS lesions is unlikely to be entirely replaced. Ideally, however, biomarkers could be used in combination with MRI for more thorough and accurate diagnosis that correlates well with clinical disability. Blood tests may be performed to assist in diagnosis, but typically only to rule out other diseases that have similar presentation.

CSF more closely reflects the events that occur in the CNS than does peripheral blood and can be expected to be a rich source of potential MS biomarkers. Although most proteins (˜80%) in the normal CSF originate from the blood, they are generally reduced 100- to 1000-fold. Blood proteins passively diffuse across capillary walls into the brain, extracellular fluid and CSF. Larger molecules exchange more slowly and have a larger concentration gradient from serum to CSF than smaller molecules. Some relative concentrations of CSF to serum are IgM (3/10,000), IgG (2/1000), and albumin (5.15/1000). Brain-derived proteins fall into 3 categories: (1) Proteins derived from neurons and glial cells such as tau, S- 100 and neuron specific enolase. These proteins are released into the ventricular and cisternal CSF where they have concentrations of 10, 18 and 1 times the levels found in serum; (2) Proteins derived primarily from the leptomeninges that are released into the CSF such as beta trace protein (prostaglandin-D-synthase) and cystatin C. The concentrations of these proteins are 30 and 5 times higher in CSF than serum, respectively; and (3) Finally, brain-derived proteins that also have a blood-derived fraction in the CSF, such as transthyretin, angiotenson converting enzyme and s-ICAM. Post-translational modifications such as glycosylation patterns may enable the origin of subsets of these proteins to be distinguished. Hoffmann, A., Nimtz, M., Getzlaff, R. & Conradt, H. S. ‘Brain-type’ N-glycosylation of asialo-transferrin from human cerebrospinal fluid. FEBS Lett 359, 164-168 (1995); Grunewald, S. et al. beta-Trace protein in human cerebrospinal fluid: a diagnostic marker for N-glycosylation defects in brain. Biochim Biophys Acta 1455, 54-60 (1999). These proteins typically have low relative concentrations of 5/100, 1/100 and 5/1000 in the CSF relative to serum. In neurological diseases with blood-CSF barrier damage all blood proteins are elevated in the CSF.

The disease course of MS is highly variable within and between patients indicating that there is disease heterogeneity. Indeed, heterogeneity in MS lesions has been shown in MRI and pathologic studies. MRI affords the ability to identify atrophy and different types of lesions, however it lacks pathologic specificity. Because of its intimate association with the CNS, considerable efforts have been made to identify prognostic and diagnostic markers in the CSF from patients with MS. To date few specific markers have been widely accepted, however several contemporary investigations have indicated that the CSF may hold many specific biomarkers for MS.

Irreversible neurological disability in MS is related, in part, to axonal damage. It can be detected on MRI as atrophy and hypointense T1 weighted lesions termed “black holes”. Pathogenic specificity is, however, lacking. Neurofilaments have been proposed as biomarkers for axonal damage in MS and other neurological diseases. Antibodies directed at neurofilaments have also been viewed as potential biomarkers. Investigators have searched for correlations between levels of these markers and clinical or MRI measures with success, albeit limited. Silber, E., Semra, Y. K., Gregson, N. A. & Sharief, M. K. Patients with progressive multiple sclerosis have elevated antibodies to neurofilament subunit. Neurology 58, 1372-1381 (2002) (clinical measures); Eikelenboom, M. J. et al. Multiple sclerosis: Neurofilament light chain antibodies are correlated to cerebral atrophy. Neurology 60, 219-223 (2003) (MRI measures).

It has been reported that individuals with MS who have intrathecal synthesis of IgM were likely to progress from relapsing remitting MS to a more severe progressive form. Villar, L. M. et al. Intrathecal IgM synthesis is a prognostic factor in multiple sclerosis. Ann Neurol 53, 222-226. (2003). This unfavorable prognostic marker appears to be maintained over several years. Patients who did not progress from the more benign course did not have detectable levels of this marker, underlining its specificity. In 82% of the patients studied with a benign form of MS this marker was absent whereas 100% with non-benign MS had the marker. The same group has also suggested that the presence of intrathecal IgM correlates with progression from initial stages of the disease to clinically definite MS, and higher EDSS scores. Villar, L. M. et al. Intrathecal IgM synthesis predicts the onset of new relapses and a worse disease course in MS. Neurology 59, 555-559. (2002); Villar, L. M. et al. Intrathecal IgM synthesis in neurologic diseases: relationship with disability in MS. Neurology 58, 824-826. (2002).

Another study evaluated biomarkers of different glial cell responses. Petzold, A. et al. Markers for different glial cell responses in multiple sclerosis: clinical and pathological correlations. Brain 125, 1462-1473. (2002). S100B is a good marker for the relapsing phase of the disease, but ferritin, which is elevated throughout the entire course, is not. Glial-fibrillary acidic protein (GFAP) correlated with disability scales and may therefore be a marker for irreversible damage.

Small molecule biomarkers have been investigated in CSF. Nitric oxide (NO) is formed in inflammatory disorders. Levels of NO, its oxidation products (NOx) and the iNOS enzyme are altered as a consequence. Several studies have examined particular parts of this response. CSF nitrite levels were correlated with disease exacerbation and concurrent inflammation in the CNS. Danilov, A. I. et al. Nitric oxide metabolite determinations reveal continuous inflammation in multiple sclerosis. J Neuroimmunol 136, 112-118. (2003). Although an important finding, the limitation of this study is that only several known components of the NO pathway were examined.

A few potential biomarkers have also been reported in serum. For example, elevated levels of CD31+ endothelial microparticles in the serum of MS patients has been reported as a potential biomarker for disease progression. Minagar, A., Jy, W., Jimenez, J. J., Sheremata, W. A., Mauro, L. M., Mao, W. W., Horstman, L. L., Ahn, Y. S. Elevated plasma endothelial microparticles in multiple sclerosis. Neurology 56, 1319-1324 (2001).

The studies described above strongly indicate that important biomarkers are present in the CSF and serum of MS patients. Some validation studies with respect to a potential biomarker have concluded particular markers are not specific for MS. Jimenez-Jimenez, F. J. et al. Tau protein concentrations in cerebrospinal fluid of patients with multiple sclerosis. Acta Neurol Scand 106, 351-354. (2002).

Proteomics and high throughput functional genomics have been applied in the investigation of MS. Gene expression profiling with DNA microarrays represents one of the major advances in functional genomics. The value of this technology in relation to MS was recently demonstrated by analysis of brain tissue. Lock, C. et al. Gene-microarray analysis of multiple sclerosis lesions yields new targets validated in autoimmune encephalomyelitis. Nat Med 8, 500-508 (2002). The expression level of numerous gene products was altered when compared to normal brain. Importantly, several of the genes identified were validated by targeting them with therapeutic approaches in experimental models of the disease. A shortcoming of gene expression profiling is the absence of information on the amount or post-transcriptional modification of the protein products. Furthermore, the analysis of products in fluids is not possible. Gene expression analysis, although useful, provides only a partial view of the biological problem of interest. On the other hand, proteomic and metabolomic approaches afford the collection of additional relevant information.

Characterization of proteins in CSF with proteomic approaches has been sparse. All of the published studies employ 2-D electrophoresis, which is rather cumbersome and typically requires more protein than routinely can be obtained with CSF. Furthermore, low-molecular-weight proteins, many other proteins, and the entire metabolome are not amenable to electrophoresis. Manabe, T. Combination of electrophoretic techniques for comprehensive analysis of complex protein systems. Electrophoresis 21, 1116-1122 (2000). Poor sensitivity has hampered some studies; others have used very large amounts of fluid to compensate. These efforts have yielded identification of a very limited number of proteins. Puchades, M., Westman, A., Blennow, K. & Davidsson, P. Analysis of intact proteins from cerebrospinal fluid by matrix-assisted laser desorption/ionization mass spectrometry after two-dimensional liquid-phase electrophoresis. Rapid Commun Mass Spectrom 13, 2450-2455 (1999). Nonetheless, employing 2-D electrophoresis proteomics and discovery driven strategies, researchers have identified biomarkers within CSF. For example, a complement factor was identified in the CSF of patients with cerebral arteriopathy. Unlu, M., de Lange, R. P., de Silva, R., Kalaria, R. & St Clair, D. Detection of complement factor B in the cerebrospinal fluid of patients with cerebral autosomal dominant arteriopathy with subcortical infarcts and leukoencephalopathy disease using two-dimensional gel electrophoresis and mass spectrometry. Neurosci Lett 282, 149-152 (2000).

Despite tremendous effort in MS research, the specific targets of the immune response, the precise mechanism for neuronal loss and the events leading to disease inception are not clear. Many investigations have examined CSF, serum and urine for such markers. Usually, one to several markers at a time have been investigated. Numerous putative MS biomarker candidates have been reported that represent different mechanisms of pathogenesis and the inflammatory cascade. Sorensen, P. S. Biological markers in body fluids for activity and progression in multiple sclerosis. Mult Scler 5, 287-290 (1999); Giovannoni, G., Green, A. J. & Thompson, E. J. Are there any body fluid markers of brain atrophy in multiple sclerosis? Mult Scler 4, 138-142 (1998). None, however, have been accepted as MS specific biomarkers. A great deal of effort is often expended toward analysis of only one potential marker at a time, resulting in failure. de Bustos, F. et al. Serum levels of coenzyme Q10 in patients with multiple sclerosis. Acta Neurol Scand 101, 209-211 (2000); Jimenez-Jimenez, F. J. et al. Cerebrospinal fluid levels of alpha-tocopherol in patients with multiple sclerosis. Neurosci Lett 249, 65-67 (1998). Other putative MS biomarkers in complex fluids have failed to be identified. Malcus-Vocanson, C. et al. Glial toxicity in urine and multiple sclerosis. Mult Scler 7, 383-388 (2001).

### SUMMARY OF THE INVENTION

The present invention provides biological markers (“biomarkers”) indicative of Multiple Sclerosis (MS). These biomarkers can be used to diagnose the disease, monitor its progression, assess response to therapy and screen drugs for treating MS. Early diagnosis and knowledge of disease progression could allow early institution of treatment when it is most appropriate and would be of the greatest benefit to the patient. In addition, such information will allow prediction of exacerbations and classification of potential MS subtypes. The ability to evaluate response to therapy will allow the personalized treatment of the disease and provided the basis for clinical trials aimed at evaluating the effectiveness of candidate drugs.

The biomarkers of the present invention include proteins and low molecular weight molecules whose measurement values in a biological sample are different (either higher or lower) in a subject with MS as compared to a standard level or reference range established by obtaining measurement values for the biomarker in subjects who do not have the disease (“normal controls”). In preferred embodiments, such difference is statistically significant. In particular, these biomarkers comprise the molecules found in CSF—Tables 1A and 1B (collectively, “Table 1”) and Table 3—and molecules found in serum—Tables 2A and 2B (collectively, “Table 2”) and Table 4 (identified in serum). Peptides or polypeptides that are at least about 70% homologous to the peptide or polypeptide markers of Tables 1-4 are also included as biomarkers.

In one embodiment, the invention provides a method for determining whether a subject has MS. In related embodiments, the invention provides a method for determining whether a subject is more likely than not to have MS, or is more likely to have MS than to have another disease. The method is performed by obtaining a biological sample, such as serum or CSF, from the subject; measuring the level of at least one of the biomarkers in the biological sample; and comparing the measured level with a standard level or reference range. Typically, the standard level or reference range is obtained by measuring the same marker or markers in a normal control or, more preferably, a set of normal controls. Depending upon the difference between the measured level and the standard level or reference range, the patient can be diagnosed as having MS, or as not having MS. As will be appreciated by one of skill in the art, a standard level or reference range is specific to the biological sample at issue. Thus, a standard level or reference range for the marker in serum that is indicative of MS would be expected to be different from the standard level or reference range (if one exists) for that same marker in CSF, urine or another tissue, fluid or compartment. Thus, references herein to measuring biomarkers will be understood to refer to measuring the level (or in some cases, the presence or absence) of the biomarker. Furthermore, references herein to comparisons between a marker measurement level and a standard level or reference range will be understood to refer to such levels or ranges for the same type of biological sample.

In another embodiment, the invention provides a method for monitoring a MS patient over time to determine whether the disease is progressing. The method is performed by obtaining a biological sample, such as serum or CSF, from the subject at a certain time (t₁); measuring the level of at least one of the biomarkers in the biological sample; and comparing the measured level with the level measured with respect to a biological sample obtained from the subject at an earlier time (t₀). Depending upon the difference between the measured levels, it can be seen whether the marker level has increased, decreased, or remained constant over the interval (t₁-t₀). Subsequent sample acquisitions and measurements can be performed as many times as desired over a range of times t₂ to tₙ. The same type of method also can be used to assess the efficacy of a therapeutic intervention in a subject where the therapy is instituted, or an ongoing therapy is changed, after t₀ and before t₁.

In another embodiment, the invention provides a method for conducting a clinical trial to determine whether a candidate drug is effective in treating MS. The method is performed by obtaining a biological sample at time t₀ from each subject in a population of subjects diagnosed with MS, and measuring the level of at least one of the biomarkers in the biological samples. Then, a dose of a candidate drug is administered to one portion or sub-population of the same subject population (“experimental group”) while a placebo is administered to the other members of the subject population (“control group”). At time t₁, after drug or placebo administration, a biological sample is acquired from the experimental and control groups and the same assays are performed on the biological samples as were previously performed to obtain measurement values. Depending upon the difference between the measured levels between the experimental and control groups, it can be seen whether the candidate drug is effective. The relative efficacy of two different drugs or other therapies for treating MS can be evaluated using this method by administering the drug or other therapy in place of the placebo. As will be apparent to one of skill in the art, the methods of the present invention may be used to evaluate an existing drug, being used to treat another indication, for its efficacy in treating MS (e.g., by comparing the efficacy of the drug relative to one currently used for treating MS in a clinical trial, as described above).

The present invention also provides molecules that specifically bind to protein and low molecular weight markers. Such marker specific reagents have utility in isolating the markers and in detecting the presence of the markers, e.g., in immunoassays.

The present invention also provides kits for diagnosing MS, monitoring progression of the disease and assessing response to therapy, the kits comprising a container for a sample collected from a subject and at least one marker specific reagent.

### DETAILED DESCRIPTION OF THE INVENTION

The present inventors have discovered biological markers whose presence and measurement levels are indicative of multiple sclerosis (MS). The biomarkers include protein and low molecular weight molecules. According to one definition, a biological marker (“biomarker”) is “a characteristic that is objectively measured and evaluated as an indicator of normal biologic processes, pathogenic processes, or pharmacological responses to therapeutic interventions.” NIH Biomarker Definitions Working Group (1998).

Biomarkers can also include patterns or ensembles of characteristics indicative of particular biological processes. The biomarker measurement can increase or decrease to indicate a particular biological event or process. In addition, if a biomarker measurement typically changes in the absence of a particular biological process, a constant measurement can indicate occurrence of that process. For more information on biomarker measurement and discovery, see U.S. patent application Ser. No. 09/558,909, “Phenotype and Biological Marker Identification System,” filed Apr. 26, 2000, herein incorporated by reference in its entirety.

In the present invention, the biomarkers are primarily used for diagnostic purposes. However they may also be used for therapeutic, drug screening and patient stratification purposes (e.g., to group patients into a number of “subsets” for evaluation).

The present invention is based on the findings of a study designed to identify biological markers for MS. Samples of CSF and serum from patients with MS were analyzed using liquid chromatography-mass spectrometry and gas chromatography-mass spectrometry, and the resulting mass spectra profiles were compared. The markers of the present invention were identified by comparing the levels measured in samples obtained from MS patients with the levels measured in samples obtained from patients who did not have the disease. Peaks consistently higher or lower in patients with MS were further investigated by using liquid chromatography mass spectrometry (or gas chromatography mass spectrometry) combined with tandem mass spectrometry techniques to identify the molecules at issue.

Measurement values of the biomarkers were found to differ in biological samples from patients with MS as compared to biological samples from normal controls. In preferred embodiments, such difference were statistically significant. Accordingly, it is believed that these biomarkers are indicators of MS.

The present invention includes all methods relying on correlations between the biomarkers described herein and the presence of MS. In a preferred embodiment, the invention provides methods for determining whether a candidate drug is effective at treating MS by evaluating the effect it has on the biomarker values. In this context, the term “effective” is to be understood broadly to include reducing or alleviating the signs or symptoms of MS, improving the clinical course of the disease, decreasing the number or severity of exacerbations, reducing the number of plaques, reducing the amount or rate of axonal demyelination, reducing the number of inflammatory cells in existing plaque or reducing in any other objective or subjective indicia of the disease. Different drugs, doses and delivery routes can be evaluated by performing the method using different drug administration conditions. The method may also be used to compare the efficacy of two different drugs or other treatments or therapies for MS.

It is expected that the biomarkers described herein will be measured in combination with other signs, symptoms and clinical tests of MS, such as MRI scans or MS biomarkers reported in the literature. Likewise, more than one of the biomarkers of the present invention may be measured in combination. Measurement of the biomarkers of the invention along with any other markers known in the art, including those not specifically listed herein, falls within the scope of the present invention.

In one embodiment, the present invention provides a method for determining whether a subject has MS. Biomarker measurements are taken of a biological sample from a patient suspected of having the disease and compared with a standard level or reference range. Typically, the standard biomarker level or reference range is obtained by measuring the same marker or markers in a set of normal controls. Measurement of the standard biomarker level or reference range need not be made contemporaneously; it may be a historical measurement. Preferably the normal control is matched to the patient with respect to some attribute(s) (e.g., age or sex). Depending upon the difference between the measured and standard level or reference range, the patient can be diagnosed as having MS or as not having MS.

What is presently referred to as MS may turn out to be a number of related, but distinguishable conditions. Indeed, four types of MS have already been recognized: (i) benign MS, (ii) relapsing remitting MS, (iii) secondary chronic progressive MS, and (iv) primary progressive MS. Additional classifications may be made, and these types may be further distinguished into subtypes. Any and all of the various forms of MS are intended to be within the scope of the present invention. Indeed, by providing a method for subsetting patients based on biomarker measurement level, the compositions and methods of the present invention may be used to uncover and define various forms of the disease.

The methods of the present invention may be used to make the diagnosis of MS, independently from other information such as the patient's symptoms or the results of other clinical or paraclinical tests. However, the methods of the present invention are preferably used in conjunction with such other data points.

Because a diagnosis is rarely based exclusively on the results of a single test, the method may be used to determine whether a subject is more likely than not to have MS, or is more likely to have MS than to have another disease, based on the difference between the measured and standard level or reference range of the biomarker. Thus, for example, a patient with a putative diagnosis of MS may be diagnosed as being “more likely” or “less likely” to have MS in light of the information provided by a method of the present invention. If a plurality of biomarkers are measured, at least one and up to all of the measured biomarkers must differ, in the appropriate direction, for the subject to be diagnosed as having (or being more likely to have) MS. Preferably, such difference is statistically significant.

The biological sample may be of any tissue or fluid. Preferably, the sample is a CSF or serum sample, but other biological fluids or tissue may be used. Possible biological fluids include, but are not limited to, plasma, urine and neural tissue. CSF represents a preferred biological sample to analyze for MS markers as it bathes the brain and removes metabolites and molecular debris from its liquid environment. Thus, biomolecules associated with the presence and/or progression of MS are expected to be present at highest concentrations in this body fluid. A CSF biomarker in itself may be particularly useful for early diagnosis of disease. Furthermore, molecules initially identified in CSF may also be present, presumably at lower concentrations, in more easily obtainable fluids such as serum and urine. Such biomarkers may be valuable for monitoring all stages of the disease and response to therapy. Serum and urine also represent preferred biological samples as they are expected to be reflective of the systemic manifestations of the disease. In some embodiments, the level of a marker may be compared to the level of another marker or some other component in a different tissue, fluid or biological “compartment.” Thus, a differential comparison may be made of a marker in CSF and serum. It is also within the scope of the invention to compare the level of a marker with the level of another marker or some other component within the same compartment.

As will be apparent to those of ordinary skill in the art, the above description is not limited to making an initial diagnosis of MS, but also is applicable to confirming a provisional diagnosis of MS or “ruling out” such a diagnosis.

As indicated in Tables 1A, 1B, 2A, 2B, 3 and 4, some of the marker measurement values are higher in samples from MS patients, while others are lower. A significant difference in the appropriate direction in the measured value of one or more of the markers indicates that the patient has (or is more likely to have) MS. If only one biomarker is measured, then that value must increase or decrease to indicate MS. If more than one biomarker is measured, then a diagnosis of MS can be indicated by a change in only one biomarker, all biomarkers, or any number in between. In some preferred embodiments, multiple markers are measured, and a diagnosis of MS is indicated by changes in multiple markers. Measurements can be of (i) a biomarker of the present invention, (ii) a biomarker of the present invention and another factor known to be associated with MS (e.g., MRI scan); (iii) a plurality of biomarkers comprising at least one biomarker of the present invention and at least one biomarker reported in the literature, or (iv) any combination of the foregoing. Furthermore, the amount of change in a biomarker level may be an indication of the relatively likelihood of the presence of the disease.

The present invention provides biomarkers that the present inventors have shown to be indicative of MS in a subject. These biomarkers are listed in Tables 1A and B (CSF proteome), 2A and B (serum proteome), 3 (CSF metabolome) and 4 (serum metabolome). Tables 1A (CSF) and 2A (serum) provide the name of the protein (also referred to herein as the “full protein”; indicated as “Protein” in the Comp. # column) along with the corresponding measured component peptide fragments with p-values of less than 0.01. Of course, other peptide fragments of such measured proteins may be obtained (by whatever means), and such other peptide fragments are included within the scope of the invention. Proteins and/or peptides for which names were not available are listed in Tables 1B (CSF) and 2B (serum). The abbreviations used in the Tables will be familiar to those of skill in the art. For clarity, in Tables 1A, 1B, 2A and 2B, “Comp. #” refers to the component number; “m/z” refers to the mass-to-charge ratio; “R.T. (min)” refers to the retention time in minutes; “z” refers to the charge; “M+H” refers to the protonated molecular ion mass; “gi #” refers to the GenInfo Identifier; “Exp. Ratio” refers to the expression ratio. In addition, in Tables 3 and 4, “RI” refers to the retention index; “Acc. Mass” refers to accurate mass (of largest mass observed permitting accurate measurement; “High Mass” refers to Largest mass observed; “Mods” refers to modifications; “DM(mD)” refers to difference in mass in milliDalton between observed and predicted values; and “DM(ppm)” refers to difference in mass in parts per million between observed and predicted values.

The methods of the present invention may be used to evaluate fragments of the listed molecules as well as molecules that contain an entire listed molecule, or at least a significant portion thereof (e.g., measured unique epitope), and modified versions of the markers. Accordingly, such fragments, larger molecules and modified versions are included within the scope of the invention.

It is to be understood that any correlations between biological sample measurements of these biomarkers and MS, as used for diagnosis of the disease or evaluating drug effect, are within the scope of the present invention.

In the methods of the invention, biomarker levels are measured using conventional techniques. A wide variety of techniques are available, including mass spectrometry, chromatographic separations, 2-D gel separations, binding assays (e.g., immunoassays), competitive inhibition assays, and so on. Any effective method in the art for measuring the level of a protein or low molecular weight marker is included in the invention. It is within the ability of one of ordinary skill in the art to determine which method would be most appropriate for measuring a specific marker. Thus, for example, a robust ELISA assay may be best suited for use in a physician's office while a measurement requiring more sophisticated instrumentation may be best suited for use in a clinical laboratory. Regardless of the method selected, it is important that the measurements be reproducible.

The markers of the invention can be measured by mass spectrometry, which allows direct measurements of analytes with high sensitivity and reproducibility. A number of mass spectrometric methods are available and could be used to accomplish the measurement. Electrospray ionization (ESI), for example, allows quantification of differences in relative concentration of various species in one sample against another; absolute quantification is possible by normalization techniques (e.g., using an internal standard). Matrix-assisted laser desorption ionization (MALDI) or the related SELDI® technology (Ciphergen, Inc.) also could be used to make a determination of whether a marker was present, and the relative or absolute level of the marker. Moreover, mass spectrometers that allow time-of-flight (TOF) measurements have high accuracy and resolution and are able to measure low abundant species, even in complex matrices like serum or CSF.

For protein markers, quantification can be based on derivatization in combination with isotopic labeling, referred to as isotope coded affinity tags (“ICAT”). In this and other related methods, a specific amino acid in two samples is differentially and isotopically labeled and subsequently separated from peptide background by solid phase capture, wash and release. The intensities of the molecules from the two sources with different isotopic labels can then be accurately quantified with respect to one another.

In addition, one- and two-dimensional gels have been used to separate proteins and quantify gels spots by silver staining, fluorescence or radioactive labeling. These differently stained spots have been detected using mass spectrometry, and identified by tandem mass spectrometry techniques.

In highly preferred embodiments, the markers are measured using mass spectrometry in connection with a separation technology, such as liquid chromatography-mass spectrometry or gas chromatography-mass spectrometry. It is particularly preferable to couple reverse-phase liquid chromatography to high resolution, high mass accuracy ESI time-of-flight (TOF) mass spectroscopy. This allows spectral intensity measurement of a large number of biomolecules from a relatively small amount of any complex biological material without sacrificing sensitivity or throughput. Analyzing a sample will allow the marker (specified by a specific retention time and m/z) to be determined and quantified.

As will be appreciated by one of skill in the art, many other separation technologies may be used in connection with mass spectrometry. For example, a vast array of separation columns are commercially available. In addition, separations may be performed using custom chromatographic surfaces (e.g., a bead on which a marker specific reagent has been immobilized). Molecules retained on the media subsequently may be eluted for analysis by mass spectrometry.

Analysis by liquid chromatography-mass spectrometry produces a mass intensity spectrum, the peaks of which represent various components of the sample, each 74component having a characteristic mass-to-charge ratio (m/z) and retention time (r.t.). The presence of a peak with the m/z and retention time of a biomarker indicates that the marker is present. The peak representing a marker may be compared to a corresponding peak from another spectrum (e.g., from a control sample) to obtain a relative measurement. Any normalization technique in the art (e.g., an internal standard) may be used when a quantitative measurement is desired. In addition, deconvoluting software is available to separate overlapping peaks. The retention time depends to some degree on the conditions employed in performing the liquid chromatography separation. The preferred conditions, and the conditions used to obtain the retention times that appear in Tables 1 and 2, are set forth in the Example 1.

The better the mass assignment, the more accurate will be the detection and measurement of the marker level in the sample. Thus, the mass spectrometer selected for this purpose preferably provides high mass accuracy and high mass resolution. The mass accuracy of a well-calibrated Micromass TOF instrument, for example, is reported to be approximately 2 mDa, with resolution m/Am exceeding 5000.

In other preferred embodiments, the level of the markers may be determined using a standard immunoassay, such as sandwiched ELISA using matched antibody pairs and chemiluminescent detection. Commercially available or custom monoclonal or polyclonal antibodies are typically used. However, the assay can be adapted for use with other reagents that specifically bind to the marker. Standard protocols and data analysis are used to determine the marker concentrations from the assay data.

A number of the assays discussed above employ a reagent that specifically binds to the marker (“marker specific reagent”). Any molecule that is capable of specifically binding to a marker is included within the invention. In some embodiments, the marker specific reagents are antibodies or antibody fragments. In other embodiments, the marker specific reagents are non-antibody species. Thus, for example, a marker specific reagent may be an enzyme for which the marker is a substrate. The marker specific reagents may recognize any epitope of the targeted markers.

A marker specific reagent may be identified and produced by any method accepted in the art. Methods for identifying and producing antibodies and antibody fragments specific for an analyte are well known. Examples of other methods used to identify marker specific reagents include binding assays with random peptide libraries (e.g., phage display) and design methods based on an analysis of the structure of the marker.

The markers of the invention, especially the low molecular weight markers, also may be detected or measured using a number of chemical derivatization or reaction techniques known in the art. Reagents for use in such techniques are known in the art, and are commercially available for certain classes of target molecules.

Finally, the chromatographic separation techniques described above also may be coupled to an analytical technique other than mass spectrometry such as fluorescence detection of tagged molecules, NMR, capillary UV, evaporative light scattering or electrochemical detection.

In an alternative embodiment of the invention, a method is provided for monitoring an MS patient over time to determine whether the disease is progressing. The specific techniques used in implementing this embodiment are similar to those used in the embodiments described above. The method is performed by obtaining a biological sample, such as serum or CSF, from the subject at a certain time (t₁); measuring the level of at least one of the biomarkers in the biological sample; and comparing the measured level with the level measured with respect to a biological sample obtained from the subject at an earlier time (t₀). Depending upon the difference between the measured levels, it can be seen whether the marker level has increased, decreased, or remained constant over the interval (t₁-t₀). A further deviation of a marker in the direction indicating MS, or the measurement of additional increased or decreased MS markers, would suggest a progression of the disease during the interval. Subsequent sample acquisitions and measurements can be performed as many times as desired over a range of times t₂ to tₙ.

The ability to monitor a patient by making serial marker level determinations would represent a valuable clinical tool. Rather than the limited “snapshot” provided by a single test, such monitoring would reveal trends in marker levels over time. In addition to indicating a progression of the disease, tracking the marker levels in a patient could be used to predict exacerbations or indicate the clinical course of the disease. For example, as will be apparent to one of skill in the art, the biomarkers of the present invention could be further investigated to distinguish between any or all of the known forms of MS (benign MS, relapsing remitting MS, secondary chronic progressive MS, and primary progressive MS) or any later described types or subtypes of the disease. In addition, the sensitivity and specificity of any method of the present invention could be further investigated with respect to distinguishing MS from other diseases of autoimmunity, or other nervous system disorders, or to predict relapse and remission.

Analogously, the markers of the present invention can be used to assess the efficacy of a therapeutic intervention in a subject. The same approach described above would be used, except a suitable treatment would be started, or an ongoing treatment would be changed, before the second measurement (i.e., after t₀ and before t₁). The treatment can be any therapeutic intervention, such as drug administration, dietary restriction or surgery, and can follow any suitable schedule over any time period. The measurements before and after could then be compared to determine whether or not the treatment had an effect effective. As will be appreciated by one of skill in the art, the determination may be confounded by other superimposed processes (e.g., an exacerbation of the disease during the same period).

In a further additional embodiment, the markers may be used to screen candidate drugs in a clinical trial to determine whether a candidate drug is effective in treating MS. At time t₀, a biological sample is obtained from each subject in population of subjects diagnosed with MS. Next, assays are performed on each subject's sample to measure levels of a biological marker. In some embodiments, only a single marker is monitored, while in other embodiments, a combination of markers, up to the total number of factors, is monitored. Next, a predetermined dose of a candidate drug is administered to a portion or sub-population of the same subject population. Drug administration can follow any suitable schedule over any time period. In some cases, varying doses are administered to different subjects within the sub-population, or the drug is administered by different routes. At time t₁, after drug administration, a biological sample is acquired from the sub-population and the same assays are performed on the biological samples as were previously performed to obtain measurement values. As before, subsequent sample acquisitions and measurements can be performed as many times as desired over a range of times t₂ to tₙ. In such a study, a different sub-population of the subject population serves as a control group, to which a placebo is administered. The same procedure is then followed for the control group: obtaining the biological sample, processing the sample, and measuring the biological markers to obtain a measurement chart.

Specific doses and delivery routes can also be examined. The method is performed by administering the candidate drug at specified dose or delivery routes to subjects with MS; obtaining biological samples, such as serum or CSF, from the subjects; measuring the level of at least one of the biomarkers in each of the biological samples; and, comparing the measured level for each sample with other samples and/or a standard level. Typically, the standard level is obtained by measuring the same marker or markers in the subject before drug administration. Depending upon the difference between the measured and standard levels, the drug can be considered to have an effect on MS. If multiple biomarkers are measured, at least one and up to all of the biomarkers must change, in the expected direction, for the drug to be considered effective. Preferably, multiple markers must change for the drug to be considered effective, and preferably, such change is statistically significant.

As will be apparent to those of ordinary skill in the art, the above description is not limited to a candidate drug, but is applicable to determining whether any therapeutic intervention is effective in treating MS.

In a typical embodiment, a subject population having MS is selected for the study. The population is typically selected using standard protocols for selecting clinical trial subjects. For example, the subjects are generally healthy, are not taking other medication, and are evenly distributed in age and sex. The subject population can also be divided into multiple groups; for example, different sub-populations may be suffering from different types or different degrees of the disorder to which the candidate drug is addressed.

In general, a number of statistical considerations must be made in designing the trial to ensure that statistically significant changes in biomarker measurements can be detected following drug administration. The amount of change in a biomarker depends upon a number of factors, including strength of the drug, dose of the drug, and treatment schedule. It will be apparent to one skilled in statistics how to determine appropriate subject population sizes. Preferably, the study is designed to detect relatively small effect sizes.

The subjects optionally may be “washed out” from any previous drug use for a suitable period of time. Washout removes effects of any previous medications so that an accurate baseline measurement can be taken. At time t₀, a biological sample is obtained from each subject in the population. Preferably, the sample is blood or CSF, but other biological fluids may be used (e.g., urine). Next, an assay or variety of assays are performed on each subject's sample to measure levels of particular biomarkers of the invention. The assays can use conventional methods and reagents, as described above. If the sample is blood, then the assays typically are performed on either serum or plasma. For other fluids, additional sample preparation steps are included as necessary before the assays are performed. The assays measure values of at least one of the biological markers described herein. In some embodiments, only a single marker is monitored, while in other embodiments, a combination of factors, up to the total number of markers, is monitored. The markers may also be monitored in conjunction with other measurements and factors associated with MS (e.g., MRI imaging). The number of biological markers whose values are measured depends upon, for example, the availability of assay reagents, biological fluid, and other resources.

Next, a predetermined dose of a candidate drug is administered to a portion or sub-population of the same subject population. Drug administration can follow any suitable schedule over any time period, and the sub-population can include some or all of the subjects in the population. In some cases, varying doses are administered to different subjects within the sub-population, or the drug is administered by different routes. Suitable doses and administration routes depend upon specific characteristics of the drug. At time t₁, after drug administration, another biological sample (the “t₁ sample”) is acquired from the sub-population. Typically, the sample is the same type of sample and processed in the same manner (for example, CSF or blood) as the sample acquired from the subject population before drug administration (the “t₀ sample”). The same assays are performed on the t₁ sample as on the t₀ sample to obtain measurement values. Subsequent sample acquisitions and measurements can be performed as many times as desired over a range of times t₂ to tₙ.

Typically, a different sub-population of the subject population is used as a control group, to which a placebo is administered. The same procedure is then followed for the control group: obtaining the biological sample, processing the sample, and measuring the biological markers to obtain measurement values. Additionally, different drugs can be administered to any number of different sub-populations to compare the effects of the multiple drugs. As will be apparent to those of ordinary skill in the art, the above description is a highly simplified description of a method involving a clinical trial. Clinical trials have many more procedural requirements, and it is to be understood that the method is typically implemented following all such requirements.

Paired measurements of the various biomarkers are now available for each subject. The different measurement values are compared and analyzed to determine whether the biological markers changed in the expected direction for the drug group but not for the placebo group, indicating that the candidate drug is effective in treating the disease. In preferred embodiments, such change is statistically significant. The measurement values at time t₁ for the group that received the candidate drug are compared with standard measurement values, preferably the measured values before the drug was given to the group, i.e., at time to. Typically, the comparison takes the form of statistical analysis of the measured values of the entire population before and after administration of the drug or placebo. Any conventional statistical method can be used to determine whether the changes in biological marker values are statistically significant. For example, paired comparisons can be made for each biomarker using either a parametric paired t-test or a non-parametric sign or sign rank test, depending upon the distribution of the data.

In addition, tests should be performed to ensure that statistically significant changes found in the drug group are not also found in the placebo group. Without such tests, it cannot be determined whether the observed changes occur in all patients and are therefore not a result of candidate drug administration.

As indicated in Tables 1 and 2, some of the marker measurement values are higher in samples from MS patients, while others are lower. The nonadjusted p-values shown were obtained by univariate analysis. A significant change in the appropriate direction in the measured value of one or more of the markers indicates that the drug is effective. If only one biomarker is measured, then that value must increase or decrease to indicate drug efficacy. If more than one biomarker is measured, then drug efficacy can be indicated by change in only one biomarker, all biomarkers, or any number in between. In some embodiments, multiple markers are measured, and drug efficacy is indicated by changes in multiple markers. Measurements can be of both biomarkers of the present invention and other measurements and factors associated with MS (e.g., measurement of biomarkers reported in the literature and/or MRI imaging). Furthermore, the amount of change in a biomarker level may be an indication of the relatively efficacy of the drug.

In addition to determining whether a particular drug is effective in treating MS, biomarkers of the invention can also be used to examine dose effects of a candidate drug. There are a number of different ways that varying doses can be examined. For example, different doses of a drug can be administered to different subject populations, and measurements corresponding to each dose analyzed to determine if the differences in the inventive biomarkers before and after drug administration are significant. In this way, a minimal dose required to effect a change can be estimated. In addition, results from different doses can be compared with each other to determine how each biomarker behaves as a function of dose.

Analogously, administration routes of a particular drug can be examined. The drug can be administered differently to different subject populations, and measurements corresponding to each administration route analyzed to determined if the differences in the inventive biomarkers before and after drug administration are significant. Results from the different routes can also be compared with each other directly.

The present invention also provides kits for diagnosing MS, monitoring progression of the disease and assessing response to therapy. The kits comprise a container for sample collected from a patient and a marker specific reagent. In developing such kits, it is within the competence of one of ordinary skill in the art to perform validation studies that would use an optimal analytical platform for each marker. For a given marker, this may be an immunoassay or mass spectrometry assay. Kit development may require specific antibody development, evaluation of the influence (if any) of matrix constituent (“matrix effects”), and assay performance specifications. It may turn out that a combination of two or more markers provides the best specificity and sensitivity, and hence utility for monitoring the disease.

Any of the methods described herein may be used in conjunction with other methods of diagnosing, monitoring and subsetting. The description of the methods herein makes reference to measuring “a marker.” Typically, however a single marker may not be sufficient to provide a definitive diagnosis of a disease.

In a preferred embodiment, the methods of the invention involve measuring two markers, more preferably three markers, and even more preferably four or more.

### EXAMPLES

The CSF biomarkers of the present invention were identified by comparing CSF samples from patients with relapsing, remitting type of MS (the “MS Group”) with CSF samples from normal healthy donors. The serum biomarkers were identified by comparing serum samples from the MS Group patients with relapsing, remitting type of MS with serum samples from normal healthy donors. The study was cross-sectional and case-controlled. All of the subjects were between 18-70 years of age. Both males and females were included.

Members of the MS group had a diagnosis of relapsing-remitting MS with a duration since diagnosis of 18 months to 5 years and an Expanded Disability Status Scale (EDSS) of 0-5.5 (or comparable evaluation if EDSS was not available). Patients were not included if they were either pregnant or had significant co-morbidity.

The CSF WBC-RBC differential, total protein and glucose for the healthy normal donors were within normal limits. Subjects were not included if they were pregnant or had a significant systemic disease by discharge diagnosis or ambulatory diagnosis or a disease expected by the investigator to affect CSF (viral or bacterial meningitis, CNS bleed, metastatic or primary CNS malignancy, etc.) by discharge diagnosis or ambulatory diagnosis. The CSF samples were collected following lumbar puncture and prepared by removing cells via centrifugation followed by aliquoting and storing at -80° C while the serum samples were allowed to clot at room temperature and were then separated and aliquoted before storing at −80° C. Example 1 describes the further processing of an exemplary sample of CSF and of serum from the MS Group. Samples obtained from the normal healthy controls were processed in the essentially the same manner.

### Example 1

Once thawed, a CSF sample from an MS patient was separated into high and low molecular weight fractions using a 5-kDa cut-off spin-filter. The further processing of these fractions is described below. The serum sample was processed differently. In order to perform metabolite analysis, 100 μL of the serum sample was added to 800 μL of a mixture of acetonitrile:acetone:water to precipitate proteins. After mixing, allowing to sit for 15 minutes, and centrifugation, the supernate was pipetted for analysis of low molecular weight components as described below. This liquid was dried under vacuum. The remaining serum sample was processed as for analysis of high molecular weight components as described below. The two fractions are also referred to herein as the “protein” or “proteome” sample and the “metabolite” or “metabolome” sample, respectively. As described below, these two fractions were processed differently.

High Molecular Weight Fraction. High abundance proteins, such as albumin and immunoglobulin, which typically dominate the high molecular weight fractions (and would decrease the dynamic range of later measurements of other components) were removed by affinity methods. The remaining proteins were enzymatically digested to generate peptide fragments. The sample (˜20 μg) was dissolved in guanidine HCl (6M), Tris buffer (pH=8.3), EDTA and dithiothreitol and incubated for about 1-2 hours to reduce and denature the protein. The sulfhydryl groups were carboxymethylated with iodoacetic acid. After buffer exchange, the desalted protein sample was incubated with trypsin overnight at 37° C.

The digest was then analyzed using reverse-phase HPLC-mass spectrometry. The reverse phase high performance (high pressure) liquid chromatography (HPLC) was physically coupled with mass spectrometry via electrospray ionization (flow rate of about 8.0 microliters/min). For the online reverse phase HPLC separation, a capillary online column packed with C18 reverse-phase material was used. Molecules retained on the RP column were eluted with increasing concentration of acetonitrile. The eluate from the column flowed into the electrospray ionization (ESI) time-of-flight (TOF) mass spectrometer (Micromass LC™; Waters Corp.) for m/z and intensity determination.

Accurate mass analysis was used to analyze the samples. Typically, about 3000 molecular components were simultaneously detected and quantified from a single injection in a two-hour analysis using liquid chromatography-mass spectrometry. A single injection was the equivalent of about 2 μL of serum, or about 100 μL of CSF, for proteins.

Low Molecular Weight Fraction. The low molecular weight fraction, comprising metabolites and free-floating peptides, was analyzed by two techniques: gas chromatography-mass spectrometry and online HPLC-mass spectrometry.

Gas chromatography mass spectrometry was used to measure volatile compounds in the low molecular weight fraction. In performing the gas chromatography mass spectrometry analysis, the CSF was first examined using a refractometer to determine the concentration; the volume was adjusted to a normalized concentration. Internal standards were also added.

Keto groups in the sample were converted to derivatizable enols using triethylammonium trifluoroacetate (TEA-TFA). Shoemaker, J. D. & Elliott, W. H. Automated screening of urine samples for carbohydrates, organic and amino acids after treatment with urease. J Chromatogr 562, 125-138 (1991). The TEA-TFA serves as the keto-enolization catalyst. The TFA derivatizes upon addition of the silylating reagent and the TEA serves as a free base in solution assisting in enolization of the keto groups in the CSF (or urine or serum). The TEA-TFA also keeps salts from precipitating during the drying process.

Volatilization was enhanced by using a silylating agent to make trimethylsilyl compounds from acidic hydrogens. The dried sample was dissolved in pyridine and N-methyl-N-trimethylsilyl-trifluoroacetamide (MSTFA) or Bis(trimethylsilyl)-trifluoroacetamide (BSTFA) and 10% TEA-TFA and then sealed under nitrogen. Heating the sample, along with the addition of pyridine, drives the reaction. The dehydration was performed via a partially automated process using a multi-sample dryer (Jones Chromatography) under flowing nitrogen.

The reaction mixture was then injected into the gas chromatograph, which was coupled to the electron-impact ionization (or chemical ionization) time-of-flight. Sample volumes of about 0.4 μL for CSF, and about 0.2 μL for serum were found to provided dense and reproducible spectra.

To analyze low molecular weight components that did not provide volatile compounds for gas chromatography-mass spectrometry (e.g., those between about 200 and 5000 Da.), reverse phase HPLC was used, coupled with mass spectrometry. Material dried after precipitation was dissolved in 0.1% aqueous formic acid and desalted on a C- 18 cartridge (Waters Corp.), dried again and redissolved in 0.1% formic acid for injection onto the liquid chromatography-mass spectrometer (flow rate of about 1 μL/min). The eluate from the column flows into the electrospray ionization (ESI) time-of-flight mass spectrometry (Micromass LCTTM; Waters Corp.) for accurate mass and charge state determination. As with peptides from digested proteins, a great many metabolite and peptide analytes were simultaneously detected and quantified by LC-mass spectrometry from a single injection.

### Example 2

### Comparison of CSF Spectra

After the differential profiling of the MS and control samples, using the procedures referred to above, a statistical comparison was made of the proteome and metabolome profiles for the two groups.

Spectra from individual samples underwent nonlinear filtering to remove noise, dynamic thresholding to separate peaks from noise and vectorized two-dimensional peak selection to take advantage of information in both the chromatography and mass-to-charge dimensions. Hastings, C. A., Norton, S. M. & Roy, S. New algorithms for processing and peak detection in liquid chromatography/mass spectrometry data. Rapid Commun Mass Spectrom 16, 462-467 (2002); Wang, C. P.; Isenhour, T. L. Time-warping algorithm applied to chromatographic peak matching gas chromatography/Fourier transform infrared/mass spectrometry. Anal. Chem. 59, 649-654 (1987). Common components in the samples were compared to enable normalization and time warping to correct for small differences in the runs. Peak lists from all of the samples were compared and a merged peak list was developed and applied for monitoring a common integrated set of peaks across all samples. This list of peaks and intensities was subject to statistical analysis and data mining. Significantly, molecular peaks can be identified across samples whether or not the structure or identity of the molecules they represent is known.

The resultant data was compared using a classical statistical analysis package. Quantitative comparison of peak intensities was done using parametric or non-parametric tests, as appropriate for each variable. Unpaired t-tests or non-parametric Wilcoxon rank sum tests were used.

### Example 3

### Identification of CSF Markers

Tandem mass spectrometry and chemical knowledge were used to identify the compounds whose concentrations were found to differ (with statistical significance) between the MS group and the control group. Where practicable, pure compounds were obtained for candidate molecules and analyzed in a similar manner to confirm or reject tentative assignments.

In a tandem mass spectrometry experiment, a target ion (precursor ion) was first isolated. To isolate a target ion, an ion trap or quadrupole-TOF mass spectrometer was used for LC-tandem mass spectrometry. The ion was then collisionally fragmented to produce a tandem mass spectrometry spectrum. This spectrum is a reproducible “fingerprint” that is characteristic of the molecule. In a protein, for example, cleavage generally occurs at specific locations on the peptide backbone. The fragmentation patterns produced by tandem mass spectrometry provide information about the molecule's structure and thereby aid in identification.

High Molecular Weight Fraction. TurboSEQUEST software (Thermo Finnigan) was used to identify peptides and proteins. Washburn, M. P., Wolters, D. & Yates, J. R. Large-scale analysis of the yeast proteome by multidimensional protein identification technology. Nat Biotechnol 19, 242-247 (2001). TurboSEQUEST uses protein databases, DNA databases, or both, to make the identification. In the case of enzymatically digested proteins, an in silico digestion of the associated proteins produces peptides with amino acid sequences theoretically revealed by a computational cleavage according to known rules; these are used to compare against the raw data. Looking up a particular molecular weight with a given mass uncertainty gives a selection of possible peptides (and hence proteins) that can give rise to those peaks. The in silico digestion can include several post-translational modifications and miscleavages. The speed of the program is improved by using an index file of pointers where each pointer indicates the start location of a subset of molecular weights. For peptides/proteins that were not in the database, de novo peptide sequencing software and BLAST searching was used. De novo peptide sequencing software is now available from several commercial sources.

TurboSEQUEST can identify up to three post-translational modifications on a peptide. Gatlin, C. L., Eng, J. K., Cross, S. T., Detter, J. C. & Yates, J. R. Automated identification of amino acid sequence variations in proteins by HPLC/microspray tandem mass spectrometry. Anal Chem 72, 757-763 (2000).

Note that among the molecular components there may be multiple peptides from the same protein (generally 2 to 4). This is useful to confirm identification. More importantly, it can facilitate the identification of post-translational modifications.

The proteins, peptides or small molecules were quantified relative to the same molecules present in a different sample, usually a control or normal sample.

The variation of the individual components of this system as measured by CVs (median) averaging 43.0% for the MS group and 36.5% for the control group for proteome measurements in CSF, and 29.0% and 28.7%, respectively for the proteome measurements in serum.

Low Molecular Weight Fraction.

The gas chromatography mass spectrometry data was analyzed with the assistance of the AMDIS computer program from National Institute of Standards and Technology (NIST). Peak selection was performed using electron-impact ionization (El) method and spectra from the NIST library of ˜100,000 compound electron-impact ionization mass spectral database. For each component, initial or confirmatory identifications were made using AMDIS' spectral matching algorithms, matching raw data against the large NIST compound library and also smaller custom libraries constructed from previously identified compounds, ˜200 purchased biochemicals, and from other studies reported in the literature. Identifications were also made in the metabolome fraction using tandem mass spectrometry in a manner similar to that described with respect to the proteome fraction.

Quantification was made relative to spiked stable-isotope-labeled compounds such as trideuterated creatine (the isotope dilution method). High confidence matching scores (greater than approximately 80% confidence) were found for many compounds.

The variation of the individual components of this system as measured by CVs (median) averaging 32.3% for the MS group and 37.0% for the control group for metabolome measurements in CSF, and 41.6% and 41.6%, respectively for the metabolome measurements in serum.

All references cited herein are fully incorporated by reference.

TABLE 1A
CSF PROTEOME (named)

| | | R.T. | | | | | | | Exp. | Fold | | |
|---|---|---|---|---|---|---|---|---|---|---|---|---|
| Comp. # | m/z | (min.) | z | M + H | Accession # | gi # | Protein Description | Peptide | Ratio | Change | Trend | P-value |
| Protein | | | | | NBHUA2 | 72059 | leucine-rich alpha-2-glycoprotein - human | | 0.47 | −2.11 | Down | 3.61 × 10⁻³ |
| 1383 | 406.72 | 35.41 | 2 | 812.43 | NBHUA2 | 72059 | leucine-rich alpha-2-glycoprotein - human | GPLQLER | 0.48 | −2.07 | Down | 4.94 × 10⁻³ |
| 4333 | 632.00 | 78.46 | 3 | 1893.98 | NBHUA2 | 72059 | leucine-rich alpha-2-glycoprotein - human | ENQLEVLEVSWLHGLK | 0.46 | −2.16 | Down | 2.29 × 10⁻³ |
| Protein | | | | | NP_000286.2 | 21361198 | serine (or cysteine) proteinase inhibitor, clade A | | 0.60 | −1.66 | Down | 9.81 × 10⁻³ |
| | | | | | | | (alpha-1 antiproteinase, antitrypsin), member 1; | | | | | |
| | | | | | | | Protease inhibitor (alpha-1-antitrypsin); protease | | | | | |
| | | | | | | | inhibitor 1 (anti-elastase), alpha-1-antitrypsin [Homo | | | | | |
| | | | | | | | sapiens] | | | | | |
| 506 | 360.17 | 26.64 | 3 | 1078.49 | NP_000286.2 | 21361198 | serine (or cysteine) proteinase inhibitor, clade A (alpha-1 | FLENEDRR | 0.51 | −1.97 | Down | 1.42 × 10⁻³ |
| | | | | | | | antiproteinase, antitrypsin), member 1; | | | | | |
| 507 | 539.76 | 26.64 | 2 | 1078.51 | NP_000286.2 | 21361198 | serine (or cysteine) proteinase inhibitor, clade A (alpha-1 | FLENEDRR | 0.46 | −2.20 | Down | 1.45 × 10⁻³ |
| | | | | | | | antiproteinase, antitrypsin), member 1; | | | | | |
| 1007 | 504.74 | 31.84 | 2 | 1008.47 | NP_000286.2 | 21361198 | serine (or cysteine) proteinase inhibitor, clade A (alpha-1 | QINDYVEK | 0.64 | −1.57 | Down | 9.78 × 10⁻³ |
| | | | | | | | antiproteinase, antitrypsin), member 1; | | | | | |
| 1364 | 343.71 | 35.21 | 2 | 686.41 | NP_000286.2 | 21361198 | serine (or cysteine) proteinase inhibitor, clade A (alpha-1 | IVDLVK | 0.69 | −1.45 | Down | 8.43 × 10⁻³ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 1521 | 444.74 | 36.82 | 2 | 888.47 | NP_000286.2 | 21361198 | serine (or cysteine) proteinase inhibitor, clade A (alpha-1 | AVLTIDEK | 0.68 | −1.48 | Down | 5.14 × 10⁻³ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 1635 | 638.34 | 37.82 | 2 | 1275.67 | NP_000286.2 | 21361198 | serine (or cysteine) proteinase inhibitor, clade A (alpha-1 | GKWERPFEVK | 0.65 | −1.53 | Down | 8.49 × 10⁻³ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 1636 | 425.89 | 37.86 | 3 | 1275.65 | NP_000286.2 | 21361198 | serine (or cysteine) proteinase inhibitor, clade A (alpha-1 | GKWERPFEVK | 0.65 | −1.54 | Down | 9.36 × 10⁻³ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 2619 | 463.62 | 47.43 | 5 | 2314.07 | NP_000286.2 | 21361198 | serine (or cysteine) proteinase inhibitor, clade A (alpha-1 | KLYHSEAFTVNFGDTEEAKK | 0.48 | −2.08 | Down | 6.74 × 10⁻⁴ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 2852 | 555.79 | 50.18 | 2 | 1110.57 | NP_000286.2 | 21361198 | serine (or cysteine) proteinase inhibitor, clade A (alpha-1 | LSITGTYDLK | 0.64 | −1.57 | Down | 7.96 × 10⁻³ |
| | | | | | | | antiproteinase, antitrypsin), member 1; | | | | | |
| 3222 | 508.30 | 54.95 | 2 | 1015.59 | NP_000286.2 | 21361198 | serine (or cysteine) proteinase inhibitor, clade A (alpha-1 | SVLGQLGITK | 0.63 | −1.58 | Down | 6.53 × 10⁻³ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 4375 | 821.43 | 80.88 | 2 | 1641.85 | NP_000286.2 | 21361198 | serine (or cysteine) proteinase inhibitor, clade A (alpha-1 | ITPNLAEFAFSLYR | 0.42 | −2.36 | Down | 6.34 × 10⁻⁴ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 4376 | 547.95 | 80.87 | 3 | 1641.83 | NP_000286.2 | 21361198 | serine (or cysteine) proteinase inhibitor, clade A (alpha-1 | ITPNLAEFAFSLYR | 0.42 | −2.38 | Down | 1.97 × 10⁻³ |
| | | | | | | | antiproteinase, antitrypsin), member 1; Protease inhibitor | | | | | |
| | | | | | | | (alpha-1-antitrypsin); protease inhibitor 1 (anti-elastase), | | | | | |
| | | | | | | | alpha-1-antitrypsin [Homo sapiens] | | | | | |
| 480 | 336.69 | 26.47 | 2 | 672.37 | NP_000362.1 | 4507725 | transthyretin (prealbumin, amyloidosis type I); | VLDAVR | 1.18 | 1.18 | Up | 1.66 × 10⁻⁵ |
| | | | | | | | Transthyretin (prealbumin) [Homo sapiens] | | | | | |
| Protein | | | | | NP_000599.1 | 4505529 | orosomucoid 2; alpha-1-acid glycoprotein, type 2 | | 0.52 | −1.92 | Down | 1.63 × 10⁻³ |
| | | | | | | | [Homo sapiens] | | | | | |
| 2810 | 412.23 | 49.79 | 3 | 1234.67 | NP_000599.1 | 4505529 | orosomucoid 2; alpha-1-acid glycoprotein, type 2 [Homo | EHVAHLLFLR | 0.52 | −1.94 | Down | 1.83 × 10⁻³ |
| | | | | | | | sapiens] | | | | | |
| 2811 | 309.42 | 49.79 | 4 | 1234.66 | NP_000599.1 | 4505529 | orosomucoid 2; alpha-1-acid glycoprotein, type 2 [Homo | EHVAHLLFLR | 0.53 | −1.89 | Down | 1.43 × 10⁻³ |
| | | | | | | | sapiens] | | | | | |
| Protein | | | | | NP_001054.1 | 4557871 | transferrin [Homo sapiens] | | 0.62 | −1.62 | Down | 2.57 × 10⁻² |
| 2025 | 373.68 | 41.67 | 4 | 1491.70 | NP_001054.1 | 4557871 | transferrin [Homo sapiens] | SKEFQLFSSPHGK | 0.48 | −2.09 | Down | 9.23 × 10⁻³ |
| 2032 | 497.91 | 41.67 | 3 | 1491.71 | NP_001054.1 | 4557871 | transferrin [Homo sapiens] | SKEFQLFSSPHGK | 0.48 | −2.08 | Down | 7.01 × 10⁻³ |
| 3751 | 815.40 | 62.64 | 2 | 1629.79 | NP_001054.1 | 4557871 | transferrin [Homo sapiens] | EDPQTFYYAVAVVK | 0.56 | −1.80 | Down | 7.79 × 10⁻³ |
| Protein | | | | | NP_001176.1 | 4502337 | alpha-2-glycoprotein 1, zinc; Alpha-2-glycoprotein, | | 0.64 | −1.55 | Down | 3.12 × 10⁻² |
| | | | | | | | zinc [Homo sapiens] | | | | | |
| 1448 | 592.62 | 36.14 | 3 | 1775.84 | NP_001176.1 | 4502337 | alpha-2-glycoprotein 1, zinc; Alpha-2-glycoprotein, zinc | QDPPSVVVTSHQAPGEK | 0.57 | −1.74 | Down | 6.35 ×10⁻³ |
| | | | | | | | [Homo sapiens] | | | | | |
| Protein | | | | | NP_002606.1 | 4505709 | serine (or cysteine) proteinase inhibitor, clade F | | 0.64 | −1.56 | Down | 2.23 × 10⁻² |
| | | | | | | | (alpha-2 antiplasmin, pigment epithelium derived | | | | | |
| | | | | | | | factor), member 1; pigment epithelium-derived | | | | | |
| | | | | | | | factor[Homo sapiens] | | | | | |
| 3058 | 548.92 | 53.47 | 3 | 1644.74 | NP_002606.1 | 4505709 | serine (or cysteine) proteinase inhibitor, clade F (alpha-2 | KTSLEDFYLDEER | 0.62 | −1.61 | Down | 2.66 × 10⁻³ |
| | | | | | | | antiplasmin, pigment epithelium derived factor), member | | | | | |
| | | | | | | | 1; pigment epithelium-derived factor [Homo sapiens] | | | | | |

TABLE 1B
CSF PROTEOME (unnamed)

| Comp. | | R.T. | | | | | Protein | | | | | |
|---|---|---|---|---|---|---|---|---|---|---|---|---|
| # | m/z | (min.) | z | M + H | Accession # | gi # | Description | Peptide | Exp. Ratio | Fold Change | Trend | P-value |
| 1262 | 304.64 | 34.51 | 2 | 608.27 | | | | | 0.31 | −3.20 | Down | 2.12 × 10⁻⁵ |
| 542 | 387.21 | 27.25 | 2 | 773.41 | | | | | 0.48 | −2.07 | Down | 8.68 × 10⁻⁴ |
| 4357 | 737.05 | 80.04 | 3 | 2209.13 | | | | | 0.60 | −1.67 | Down | 1.18 × 10−3 |
| 4044 | 557.81 | 68.75 | 2 | 1114.61 | | | | | 0.63 | −1.60 | Down | 1.55 × 10−3 |
| 4041 | 372.21 | 68.74 | 3 | 1114.61 | | | | | 0.67 | −1.49 | Down | 1.61 × 10−3 |
| 2576 | 615.83 | 47.51 | 2 | 1230.65 | | | | | 0.53 | −1.89 | Down | 1.83 × 10−3 |
| 4189 | 607.30 | 72.94 | 2 | 1213.59 | | | | | 0.53 | −1.89 | Down | 2.04 × 10−3 |
| 1527 | 309.48 | 36.80 | 3 | 926.42 | | | | | 0.77 | −1.30 | Down | 2.05 × 10−3 |
| 846 | 439.74 | 30.14 | 2 | 878.47 | | | | | 0.57 | −1.77 | Down | 2.11 × 10−3 |
| 233 | 437.71 | 22.98 | 2 | 874.41 | | | | | 0.51 | −1.95 | Down | 2.63 × 10−3 |
| 556 | 429.20 | 27.21 | 2 | 857.39 | | | | | 0.35 | −2.83 | Down | 2.80 × 10−3 |
| 397 | 509.27 | 25.57 | 2 | 1017.53 | | | | | 2.01 | 2.01 | Up | 2.91 × 10−3 |
| 2986 | 501.88 | 51.93 | 3 | 1503.62 | | | | | 0.58 | −1.72 | Down | 3.42 × 10−3 |
| 4324 | 414.69 | 77.42 | 2 | 828.37 | | | | | 2.26 | 2.26 | Up | 3.43 × 10−3 |
| 656 | 320.79 | 28.50 | 3 | 960.35 | | | | | 0.78 | −1.28 | Down | 3.64 × 10−3 |
| 4225 | 556.30 | 74.18 | 2 | 1111.59 | | | | | 1.94 | 1.94 | Up | 3.88 × 10−3 |
| 296 | 450.21 | 24.02 | 2 | 899.41 | | | | | 0.41 | −2.46 | Down | 4.05 × 10−3 |
| 1597 | 436.19 | 37.45 | 3 | 1306.55 | | | | | 0.53 | −1.89 | Down | 4.22 × 10−3 |
| 1605 | 414.90 | 37.61 | 3 | 1242.68 | | | | | 0.58 | −1.71 | Down | 4.51 × 10−3 |
| 3195 | 378.87 | 54.74 | 3 | 1134.59 | | | | | 0.46 | −2.15 | Down | 4.56 × 10−3 |
| 70 | 529.72 | 16.54 | 2 | 1058.43 | | | | | 0.49 | −2.03 | Down | 4.65 × 10−3 |
| 3188 | 567.81 | 54.72 | 2 | 1134.61 | | | | | 0.37 | −2.70 | Down | 4.66 × 10−3 |
| 2985 | 752.33 | 51.93 | 2 | 1503.65 | | | | | 0.53 | −1.90 | Down | 5.42 × 10−3 |
| 3057 | 401.23 | 53.37 | 2 | 801.45 | | | | | 0.46 | −2.20 | Down | 5.50 × 10−3 |
| 1228 | 634.32 | 34.15 | 2 | 1267.63 | | | | | 1.83 | 1.83 | Up | 6.05 × 10−3 |
| 3581 | 526.21 | 60.05 | 2 | 1051.41 | | | | | 2.23 | 2.23 | Up | 6.07 × 10−3 |
| 287 | 397.20 | 24.00 | 2 | 793.39 | | | | | 0.35 | −2.82 | Down | 6.25 × 10−3 |
| 1637 | 329.92 | 37.86 | 4 | 1316.66 | | | | | 0.69 | −1.46 | Down | 6.37 × 10−3 |
| 1166 | 374.68 | 34.16 | 2 | 748.35 | | | | | 1.86 | 1.86 | Up | 6.56 × 10−3 |
| 3115 | 590.26 | 54.11 | 2 | 1179.51 | | | | | 2.28 | 2.28 | Up | 6.81 × 10−3 |
| 3737 | 475.73 | 62.41 | 4 | 1899.90 | | | | | 0.30 | −3.30 | Down | 6.86 × 10−3 |
| 4323 | 828.39 | 77.43 | 1 | 828.39 | | | | | 2.00 | 2.00 | Up | 7.79 × 10−3 |
| 2896 | 316.42 | 50.86 | 4 | 1262.66 | | | | | 0.47 | −2.13 | Down | 8.26 × 10−3 |
| 4088 | 670.31 | 70.38 | 2 | 1339.61 | | | | | 0.69 | −1.44 | Down | 8.28 × 10−3 |
| 1758 | 900.51 | 39.14 | 1 | 900.51 | | | | | 0.49 | −2.02 | Down | 8.66 × 10−3 |
| 140 | 525.70 | 21.17 | 2 | 1050.39 | | | | | 0.79 | −1.26 | Down | 8.80 × 10−3 |
| 98 | 393.22 | 20.04 | 2 | 785.43 | | | | | 0.65 | −1.54 | Down | 8.82 × 10−3 |
| 1384 | 351.71 | 35.40 | 2 | 702.41 | | | | | 0.66 | −1.50 | Down | 9.67 × 10−3 |
| 498 | 344.68 | 26.51 | 2 | 688.35 | | | | | 0.70 | −1.43 | Down | 9.86 × 10−3 |
| 4192 | 901.81 | 73.29 | 3 | 2703.41 | | | | | 0.36 | −2.78 | Down | 9.99 × 10−3 |

TABLE 2A
SERUM PROTEOME (components named)

| Comp. | | R.T. | | | | | | | Exp. | Fold | | |
|---|---|---|---|---|---|---|---|---|---|---|---|---|
| # | m/z | (min.) | z | M + H | Accession # | gi # | Protein Description | Peptide | Ratio | Change | Trend | P-value |
| Protein | | | | | NP_000005.1 | 4557225 | alpha 2 macroglobulin precursor | | | | | |
| | | | | | | | [Homo sapiens] | | | | | |
| 219 | 765.33 | 20.64 | 2 | 1529.65 | NP_000005.1 | 4557225 | alpha 2 macroglobulin precursor | TAQEGDHGSHVYTK | 1.49 | 1.49 | Up | 7.17 × 10−4 |
| | | | | | | | [Homo sapiens] | | | | | |
| Protein | | | | | NP_000629.2 | 18201911 | vitronectin precursor; serum | | 0.76 | −1.31 | Down | 1.94 × 10−2 |
| | | | | | | | spreading factor; somatomedin | | | | | |
| | | | | | | | B; complement S-protein [Homo | | | | | |
| | | | | | | | sapiens] | | | | | |
| 3047 | 474.88 | 46.97 | 3 | 1422.62 | NP_000629.2 | 18201911 | vitronectin precursor; serum | FEDGVLDPDYPR | 0.77 | −1.31 | Down | 1.41 × 10⁻³ |
| | | | | | | | spreading factor; somatomedin B; | | | | | |
| | | | | | | | complement S-protein [Homo | | | | | |
| | | | | | | | sapiens] | | | | | |
| Protein | | | | | NP_005134.1 | 4826762 | haptoglobin [Homo sapiens] | | 1.32 | 1.32 | Up | 2.13 × 10−2 |
| 3507 | 859.36 | 50.32 | 4 | 3434.42 | NP_005134.1 | 4826762 | haptoglobin [Homo sapiens] | AVGDKLPEC*EADDGC*PKPPEIAHGYVEHSVR | 1.37 | 1.37 | Up | 8.81 × 10⁻³ |
| Protein | | | | | NP_443204.1 | 16418467 | leucine-rich alpha-2-glycoprotein | | 1.35 | 1.35 | Up | 5.87 × 10⁻³ |
| | | | | | | | [Homo sapiens] | | | | | |
| 1557 | 406.73 | 35.01 | 2 | 812.45 | NP_443204.1 | 16418467 | leucine-rich alpha-2-glycoprotein | GPLQLER | 1.39 | 1.39 | Up | 5.62 × 10−4 |
| | | | | | | | [Homo sapiens] | | | | | |
| 1958 | 384.87 | 38.73 | 3 | 1152.59 | NP_443204.1 | 16418467 | leucine-rich alpha-2-glycoprotein | ALGHLDLSGNR | 1.31 | 1.31 | Up | 5.64 × 10⁻³ |
| | | | | | | | [Homo sapiens] | | | | | |
| 5768 | 679.67 | 73.00 | 3 | 2036.99 | NP_443204.1 | 16418467 | leucine-rich alpha-2-glycoprotein | TLDLGENQLETLPPDLLR | 1.32 | 1.32 | Up | 2.03 × 10⁻³ |
| | | | | | | | [Homo sapiens] | | | | | |
| 6058 | 631.98 | 78.94 | 3 | 1893.92 | NP_443204.1 | 16418467 | leucine-rich alpha-2-glycoprotein | ENQLEVLEVSWLHGLK | 1.38 | 1.38 | Up | 1.05 × 10⁻³ |
| | | | | | | | [Homo sapiens] | | | | | |
| Protein | | | | | NP_653247.1 | 21489959 | immunoglobulin J polypeptide, | | 1.35 | 1.35 | Up | 7.15 × 10⁻³ |
| | | | | | | | linker protein for immunoglobulin | | | | | |
| | | | | | | | alpha and mu polypeptides | | | | | |
| | | | | | | | [Homo sapiens] | | | | | |
| 3671 | 428.20 | 51.79 | 3 | 1282.58 | NP_653247.1 | 21489959 | immunoglobulin J polypeptide, | FVYHLSDLC*K | 1.35 | 1.35 | Up | 7.15 × 10⁻³ |
| | | | | | | | linker protein for immunoglobulin | | | | | |
| | | | | | | | alpha and mu polypeptides [Homo | | | | | |
| | | | | | | | sapiens] | | | | | |
| Protein | | | | | P01860 | 121045 | GC3_HUMAN Ig gamma-3 chain | | | | | |
| | | | | | | | C region (Heavy chain disease | | | | | |
| | | | | | | | protein) (HDC) | | | | | |
| 5803 | 718.03 | 74.17 | 3 | 2152.07 | P01860 | 121045 | GC3_HUMAN Ig gamma-3 chain C | C*PAPELLGGPSVFLFPPKPK | 0.73 | −1.36 | Down | 8.66 × 10⁻³ |
| | | | | | | | region (Heavy chain disease | | | | | |
| | | | | | | | protein) (HDC) | | | | | |
| Protein | | | | | P01871 | 127514 | MUC_HUMAN Ig mu chain C | | 1.51 | 1.51 | Up | 3.58 × 10−2 |
| | | | | | | | region | | | | | |
| 3973 | 695.05 | 54.15 | 4 | 2777.18 | P01871 | 127514 | MUC_HUMAN Ig mu chain C region | YAATSQVLLPSKDVMQGTDEHVVC*K | 1.69 | 1.69 | Up | 3.07 × 10⁻³ |
| Protein | | | | | S22657 | 7438758 | Ig heavy chain precursor V | | 0.48 | −2.09 | Down | 4.97 × 10−4 |
| | | | | | | | region (0-81VH) - human | | | | | |
| | | | | | | | (fragment) | | | | | |
| 1605 | 435.84 | 35.67 | 3 | 1305.50 | S22657 | 7438758 | Ig heavy chain precursor V region | ADDTAVYYC*AR | 0.48 | −2.09 | Down | 4.97 × 10−4 |
| | | | | | | | (0-81VH) - human (fragment) | | | | | |

TABLE 2B
SERUM PROTEOME (not named)

| | | R.T. | | | | | Protein | | | Fold | | |
|---|---|---|---|---|---|---|---|---|---|---|---|---|
| Comp. # | m/z | (min.) | z | M + H | Accession # | gi # | Description | Peptide | Exp. Ratio | Change | Trend | P-value |
| 3166 | 518.24 | 48.39 | 3 | 1552.70 | | | | | 1.46 | 1.46 | Up | 1.25 × 10⁻³ |
| 3344 | 462.20 | 49.66 | 3 | 1384.58 | | | | | 1.24 | 1.24 | Up | 1.44 × 10⁻³ |
| 6470 | 969.63 | 90.58 | 4 | 3875.50 | | | | | 1.71 | 1.71 | Up | 1.49 × 10⁻³ |
| 101 | 312.66 | 14.04 | 2 | 624.31 | | | | | 0.74 | −1.36 | Down | 1.69 × 10⁻³ |
| 246 | 441.70 | 21.51 | 2 | 882.39 | | | | | 1.50 | 1.50 | Up | 1.73 × 10⁻³ |
| 1243 | 413.17 | 32.46 | 2 | 825.33 | | | | | 1.33 | 1.33 | Up | 2.84 × 10⁻³ |
| 1202 | 400.75 | 32.26 | 2 | 800.49 | | | | | 1.52 | 1.52 | Up | 3.51 × 10⁻³ |
| 740 | 650.79 | 27.88 | 2 | 1300.57 | | | | | 1.34 | 1.34 | Up | 4.97 × 10⁻³ |
| 747 | 630.34 | 27.77 | 1 | 630.34 | | | | | 0.82 | −1.21 | Down | 7.33 × 10⁻³ |
| 658 | 531.27 | 26.87 | 1 | 531.27 | | | | | 0.68 | −1.47 | Down | 7.59 × 10⁻³ |
| 3513 | 517.57 | 50.29 | 3 | 1550.69 | | | | | 1.37 | 1.37 | Up | 8.53 × 10⁻³ |
| 1154 | 335.57 | 32.00 | 5 | 1673.82 | | | | | 1.39 | 1.39 | Up | 8.91 × 10⁻³ |
| 2856 | 479.45 | 45.23 | 4 | 1914.78 | | | | | 0.62 | −1.62 | Down | 9.27 × 10⁻³ |

TABLE 3
CSF METABOLOME

| Comp. | | R.T. | | | | NIST | Molecular | Acc. |
|---|---|---|---|---|---|---|---|---|
| # | RI | (min.) | z | M + H | Accession # | Score | Description | Mass |
| 129 | 1528.00 | 41.37 | 1 | 1528.00 | 1.00 | | | 498.137 |
| 1384 | 1155.70 | 19.27 | 1 | 1155.70 | 2.00 | 85 | 3-Hydroxybutyric | |
| | | | | | | | acid | |
| 1411 | 1808.20 | 52.95 | 1 | 1808.20 | 3.00 | 89 | D-Fructose | |
| 1179 | 1019.20 | 10.86 | 1 | 1019.20 | 4.00 | | | 172.060 |
| 570 | 1947.50 | 57.64 | 1 | 1947.50 | 5.00 | 66 | Inositol | |
| 461 | 1165.10 | 19.40 | 1 | 1165.10 | 6.00 | 91 | 2-Hydroxy | |
| | | | | | | | Pentanoic Acid | |
| 409 | 2636.00 | 70.11 | 1 | 2636.00 | 8.00 | 66 | Maltose | |
| 1381 | 1120.80 | 16.98 | 1 | 1120.80 | 9.00 | 71 | a- | |
| | | | | | | | Hydroxyisobutyric | |
| | | | | | | | acid | |
| 1046 | 2058.00 | 60.21 | 1 | 2058.00 | 10.00 | | | 539.243 |
| 1005 | 1700.30 | 49.14 | 1 | 1700.30 | 11.00 | | | 332.127 |

| Comp. | High | | | | | Exp | Fold | |
|---|---|---|---|---|---|---|---|---|
| # | Mass | Mods | DM(mD) | DM(ppm) | Ratio | Change | Trend | P-value |
| 129 | 498 | | 0.0 | 0.0 | 0.50 | −2.02 | Down | 3.94 × 10−4 |
| 1384 | | | 0.0 | 0.0 | 0.57 | −1.74 | Down | 1.65 × 10⁻³ |
| 1411 | | | 0.0 | 0.0 | 1.25 | 1.25 | Up | 1.92 × 10⁻³ |
| 1179 | 172 | | 0.0 | 0.0 | 1.22 | 1.22 | Up | 3.16 × 10⁻³ |
| 570 | | | 0.0 | 0.0 | 0.44 | −2.29 | Down | 3.41 × 10⁻³ |
| 461 | | | 0.0 | 0.0 | 0.79 | −1.26 | Down | 4.94 × 10⁻³ |
| 409 | | | 0.0 | 0.0 | 0.52 | −1.92 | Down | 5.51 × 10⁻³ |
| 1381 | | | 0.0 | 0.0 | 0.73 | −1.38 | Down | 5.57 × 10⁻³ |
| 1046 | 539 | | 0.0 | 0.0 | 0.56 | −1.79 | Down | 7.87 × 10⁻³ |
| 1005 | 332 | | 0.0 | 0.0 | 0.60 | −1.66 | Down | 8.91 × 10⁻³ |

TABLE 4
SERUM METABOLOME

| | | R.T. | | | | NIST | Molecular | Acc. |
|---|---|---|---|---|---|---|---|---|
| Comp. # | RI | (min.) | z | M + H | Accession # | Score | Description | Mass |
| 488 | 1899.60 | | | 1.01 | 1 | 89.3 | alpha-D- | |
| | | | | | | | Manno- | |
| | | | | | | | pyranose | |
| 949 | 2896.10 | | | 1.01 | 2 | | | 428.402 |

| | | | | | | Exp. | Fold | |
|---|---|---|---|---|---|---|---|---|
| Comp. # | High Mass | Mods | DM(mD) | DM(ppm) | Ratio | Change | Trend | P value |
| 488 | | | 0.0 | 0.0 | 1.44 | 1.44 | Up | 3.97 × 10⁻³ |
| 949 | | | 0.0 | 0.0 | 1.48 | 1.48 | Up | 5.03 × 10⁻³ |

1. A method for diagnosing multiple sclerosis in a subject, the method comprising:
obtaining a biological sample from the subject;
determining the level of a marker in the sample, wherein the marker is selected from the group consisting of the molecules set forth in Tables 1-4; and
comparing the level of the marker in the sample to a reference value.

2. The method of claim 1, wherein the biological sample is a body fluid.

3. The method of claim 2, wherein the body fluid is selected from the group consisting of blood, serum, plasma, cerebrospinal fluid, urine, and saliva.

4. The method of claim 1, wherein the marker comprises a polypeptide or fragment thereof.

5. The method of claim 1, wherein the marker comprises a metabolite or fragment thereof.

6. The method of claim 1, wherein the marker is selected from the group consisting of the molecules set forth in Tables 1 and 2.

7. The method of claim 6, wherein the marker is selected from the group consisting of the molecules set forth in Tables 1A and 2A.

8. The method of claim 7, wherein the marker is selected from the group consisting of the full proteins set forth in Tables 1 A and 2A or fragment thereof.

9. The method of claim 1, wherein the marker the marker is selected from the group consisting of the molecules set forth in Tables 3 and 4.

10. The method of claim 1, wherein the reference value is the level of the marker in at least one sample from a non-multiple sclerosis subject.

11. A method for diagnosing multiple sclerosis in a subject, the method comprising:
obtaining one or more biological samples from the subject;
determining the level of a plurality of markers in the one or more biological samples, wherein at least one of the plurality of markers is selected from the group consisting of the molecules disclosed in Tables 1-4;
comparing the level of at least one of the plurality of markers to a reference value.

12. The method of claim 11, wherein the biological sample is a body fluid.

13. The method of claim 12, wherein the body fluid is selected from the group consisting of blood, serum, plasma, cerebrospinal fluid, urine, and saliva.

14. The method of claim 11, wherein at least one of the plurality of markers is a polypeptide or a fragment thereof.

15. The method of claim 1 1, wherein at least one of the plurality of markers is a metabolite or a fragment thereof.

16. The method of claim 11, wherein at least one of the plurality of markers is a metabolite or a fragment thereof and at least one of the plurality of markers is a protein or a fragment thereof.

17. The method of claim 1 1, wherein at least two of the plurality of markers are selected from the group consisting of the molecules set forth in Tables 1-4.

18. The method of claim 11, wherein at least ten of the plurality of markers are selected from the group consisting of the molecules set forth in Tables 1-4.

19. The method of claim 1 1, wherein at least one of the plurality of markers is selected from the group consisting of molecules set forth in Tables 1-2.

20. The method of claim 19, wherein the reference value is the level of at least one of the plurality of markers in at least one sample from a non-multiple sclerosis subject, and wherein the level of the at least one of the plurality of markers is increased by at least one fold with respect to the reference value.

21. The method of claim 20, wherein the level of the at least one of the plurality of markers is increased by at least two fold with respect to the reference value.

22. The method of claim 11, wherein at least one of the plurality of markers is selected from the group consisting of the molecules set forth in Tables 3-4.

23. The method of claim 22, wherein the reference value is the level of the at least one of the plurality of markers in at least one sample from a non-multiple sclerosis subject, and wherein the level of the at least one of the plurality of markers is increased by at least one fold with respect to the reference value.

24. The method of claim 23, wherein the level of the at least one of the plurality of markers is increased by at least two fold with respect to the reference value.

25. The method of claim 1, wherein the marker is not expressed in non-multiple sclerosis subjects.

26. The method of claim 1, wherein the level of the marker is determined by detecting the presence of a polypeptide.

27. The method of claim 26, wherein the polypeptide is the marker.

28. The method of claim 26, wherein the polypeptide shares 70% homology with the marker.

29. The method of claim 26, wherein the polypeptide is a modified form of the marker.

30. The method of claim 26, wherein the polypeptide is a precursor to the marker.

31. The method of claim 26, wherein the polypeptide is a metabolite of the marker.

32. The method of claim 26, wherein the method further comprises detecting the presence of the polypeptide using a reagent that specifically binds to the polypeptide or a fragment thereof.

33. The method of claim 32, wherein the reagent is selected from the group consisting of an antibody, an antibody derivative, and an antibody fragment.

34. The method of claim 1, wherein the level of the marker is determined by detecting the presence of a metabolite.

35. The method of claim 34, wherein the metabolite is the marker.

36. The method of claim 34, wherein the metabolite is a modified form of the marker.

37. The method of claim 34, wherein the metabolite is a precursor to the marker.

38. The method of claim 34, wherein the metabolite is a metabolic product of the marker.

39. The method of claim 1, wherein the subject is a lab animal.

40. The method of claim 1, wherein the subject is a human subject.

41. A method for monitoring the progression of multiple sclerosis in a subject, the method comprising:
obtaining a first biological sample from the subject;
measuring the level of a marker in the first sample, wherein the marker is selected from the group consisting of the molecules set forth in Tables 1-4;
obtaining a second biological sample from the subject;
measuring the level of the marker in the second sample; and
comparing the level of the marker measured in the first sample with the level of the marker measured in the second sample.

42. A method of assessing the efficacy of a treatment for multiple sclerosis in a subject, the method comprising comparing:
(i) the level of a marker measured in a first sample obtained from the subject before the treatment has been administered to the subject, wherein the marker is selected from the group consisting of the molecules set forth in Tables 1-2; and
(ii) the level of the marker in a second sample obtained from the subject after the treatment has been administered to the subject,
wherein a decrease in the level of the marker in the second sample relative to the first sample is an indication that the treatment is efficacious for treating multiple sclerosis in the subject.

43. A method of assessing the efficacy of a treatment for multiple sclerosis in a subject, the method comprising comparing:
(i) the level of a marker in a first sample obtained from the subject before the treatment has been administered to the subject, wherein the marker is selected from the group consisting of the molecules set forth in Tables 3-4; and
(ii) the level of the marker in a second sample obtained from the subject after the treatment has been administered to the subject,
wherein an increase in the amount of the marker in the second sample, relative to the first sample, is an indication that the treatment is efficacious for inhibiting multiple sclerosis in the subject.

44. A method of treating multiple sclerosis in a subject, the method comprising inhibiting expression of a gene corresponding to a marker selected from the group consisting of the molecules set forth in Tables 1-4.

45. A method for diagnosing multiple sclerosis in a subject, the method comprising:
obtaining a biological sample from a subject;
determining a first amount of a first marker in the biological sample, wherein the first marker is increased in subjects with multiple sclerosis;
determining a second amount of a second marker in the biological sample, wherein the second marker is decreased in subjects with multiple sclerosis;
comparing the first amount to a first reference value and comparing the second amount to a second reference value, wherein a significantly difference exists
[As used herein, a “significantly different is one that permits the other protein to be resolved] between both (i) the first amount and the first reference value and (ii) second amount and the second reference value, and wherein the differences are indicative that the subject has multiple sclerosis.

46. The method of claim 45, wherein the first marker is a molecule selected from the group consisting of the molecules set forth in Tables 1-2.

47. The method of claim 45, wherein the second marker is a molecule selected from the group consisting of the molecules set forth in Tables 3-4.

48. A method for diagnosing multiple sclerosis in a subject, the method comprising:
obtaining a sample from the subject;
determining the amount of at least one first marker in the sample, wherein the at least one first marker is selected from the group consisting of the molecules set forth in Tables 1-2;
determining the amount of at least one second marker in the sample, wherein the at least one second marker is selected from the group consisting of the molecules set forth in Tables 3-4;
comparing the amounts of the at least one first marker and at least one second marker in the sample from the subject to the amounts of the at least one first marker and at least one second marker in at least one sample from a subject not suspected of having multiple sclerosis, wherein a measurable difference exists between the amounts measured for at least 50% of the markers.

49. An isolated molecule selected from the group consisting of the molecules set forth in Tables 1-4.

50. A composition comprising a molecule selected from the group consisting of the molecules set forth in Tables 1-4.

51. A method for aiding in the diagnosis of multiple sclerosis in a subject, the method comprising:
obtaining a biological sample from the subject;
determining the level of a marker in the sample, wherein the marker is selected from the group consisting of the molecules set forth in Tables 1-4;
comparing the level of the marker in the sample to a reference value; and
determining from the results of the comparison whether the subject is more or less likely to have multiple sclerosis.

52. A method for determining the type, stage or severity of multiple sclerosis in a subject, the method comprising:
obtaining a biological sample from the subject;
determining the level of a marker in the sample, wherein the marker is selected from the group consisting of the molecules set forth in Tables 1-4;
comparing the level of the marker in the sample to a reference value; and
determining from the results of the comparison the type, stage or severity of multiple sclerosis in the subject.

53. A method for determining the risk of developing multiple sclerosis in a subject, the method comprising:
obtaining a biological sample from the subject;
determining the level of a marker in the sample, wherein the marker is selected from the group consisting of the molecules set forth in Tables 1-4;
comparing the level of the marker in the sample to a reference value; and
determining from the results of the comparison that the subject has an increased or decreased risk of developing multiple sclerosis.

54. The method of claim 1, wherein the marker shares 70% homology with one or more of the molecules set forth in Tables 1-4.

55. The method of claim 1, wherein the marker is a modified form of one or more of the molecules set forth in Tables 1-4.

56. The method of claim 1, wherein the marker is a precursor to one or more of the molecules set forth in Tables 1-4.

57. The method of claim 1, wherein the marker is a metabolite of one or more of the molecules set forth in Tables 1-4.

58. The method of claim 1, wherein the marker is a compound in a known metabolic pathway including one or more of the molecules set forth in Tables 1-4.

59. The method of claim 1, wherein the marker-regulates a known metabolic pathway including one or more of the molecules set forth in Tables 1-4.

60. A kit comprising a molecule selected from the group consisting of the molecules set forth in Tables 1-4.

61. A kit comprising a reagent that specifically binds to a molecule selected from the group consisting of the molecules set forth in Tables 1-4.

62. A method for diagnosing multiple sclerosis in a subject, the method comprising:
obtaining a biological sample from the subject;
determining the level of a protein in the sample that specifically binds to a marker, wherein the marker is selected from the group consisting of set forth in Tables 1-4; comparing the level of the protein marker in the sample to a reference value.
